(19) United States
(12) Patent Application Publication
(10) Pub. No.: US 2024/0382745 A1
(43) Pub. Date: Nov. 21, 2024

(54) IMPLANTABLE PATIENT-CONTROLLED ANALGESIC DEVICE

(71) Applicant: University of Washington, Seattle, WA (US)

(72) Inventors: Rajiv Saigal, Danville, CA (US); Erin Graf, San Francisco, CA (US); Abigail Green, Seattle, WA (US)

(73) Assignee: University of Washington, Seattle, WA (US)

(21) Appl. No.: 18/256,851

(22) PCT Filed: Dec. 10, 2021
(86) PCT No.: PCT/US2021/062940

§ 371 Date: Jun. 9, 2023

Related U.S. Application Data
(60) Provisional application No. 63/125,229, filed on Dec. 14, 2020.

(51) Int. Cl. A61N 1/05; A61N 1/36

(52) U.S. Cl. CPC A61N 1/0558; A61N 1/36071

(57) ABSTRACT

An implantable patient-controlled analgesic device includes a microelectrode, a control member, and a connections member. The microelectrode comprises a conductive polymer doped with a dopant, wherein the microelectrode is configured to be implanted within a patient's body. The control member is configured to control the microelectrode via an electrical current. The connection member is coupled to the microelectrode and the control member such that the electrical current can flow from the control member to the microelectrode. In some instances, the implantable patient-controlled analgesic device can be coupled to a surgical drain.

### CROSS REFERENCE TO RELATED APPLICATION

This application claims the benefit of the earlier filing date of U.S. Provisional Application No. 63/125,229, filed on Dec. 14, 2020, the entirety of which is incorporated by reference herein.

### FIELD

The present disclosure is directed to devices and methods for pain management and, more particularly, patient-controlled analgesic devices.

### BACKGROUND

Appropriate management of pain, which attenuates the body's stress response, is a vital aspect of enhanced recovery and long-term quality of life following injury and/or a medical procedure. Oral and intravenous opioids are commonly used to manage pain. While opioids have been foundational to analgesia for many years, physicians have long questioned the use of opioids given their adverse side effects and propensity for addiction. Current alternatives to oral opioids include local anesthetic injection, local anesthetic implanted pumps, and liposomal-encased anesthetics. While these current solutions address acute pain relief, they add unnecessary steps, while increasing recovery time and risk of infection or increasing cost. As such, there is a continuing need for improved devices and methods for managing pain.

### SUMMARY

Disclosed herein are devices and methods for managing pain that overcome or reduce the shortcomings of typical pain management devices and methods. For example, the disclosed devices and methods can reduce the need for opioids and optimize pain management.

In one representative example, an implantable patient-controlled analgesic device comprises a microelectrode, a control member, and a connections member. The microelectrode comprises a conductive polymer doped with a dopant, wherein the microelectrode is configured to be implanted within a patient's body. The control member is configured to control the microelectrode via an electrical current. The connection member is coupled to the microelectrode and the control member such that the electrical current can flow from the control member to the microelectrode.

In some examples, the dopant comprises one or more of bupivacaine, a non-steroidal anti-inflammatory drug, an opioid, and/or or other anesthetic or analgesic.

In some examples, the implantable patient-controlled analgesic device further comprises a main body on which the microelectrode is disposed.

In some examples, the microelectrode is one microelectrode of a plurality of microelectrodes arranged in an array.

In some examples, the implantable patient-controlled analgesic device further comprises a surgical drain portion, the surgical drain portion comprises a plurality of openings and a conduit, and the conduit is in fluidic communication with the plurality of openings such that fluid can flow into the openings and through the conduit. In some such examples, the surgical drain portion is disposed distal relative to the microelectrode. In other such examples, the surgical drain portion is disposed proximal relative to the microelectrode.

In some examples, the microelectrode comprises one or more of polyimide and poly-dimethyl-siloxane.

In some examples, the conductive polymer comprises poly-pyrrole.

In some examples, the microelectrode comprises one or more of titanium, platinum, gold, and chromium.

In another representative example, an assembly comprises a medical implant and an implantable patient-controlled analgesic device coupled to the medical implant. The implantable patient-controlled analgesic device coupled to the medical implant includes a microelectrode comprising a conductive polymer doped with a dopant, and the microelectrode is configured to be implanted within a patient's body. The control member is configured to control the microelectrode via an electrical current. The connection member is coupled to the microelectrode and the control member such that the electrical current can flow from the control member to the microelectrode.

In some examples, the medical implant comprises a surgical drain having a main body with a plurality of openings disposed at a distal end portion, and the microelectrode comprises an array of microelectrodes disposed at a proximal end portion of the main body.

In some examples, the medical implant comprises a surgical drain having a main body with a plurality of openings disposed at a proximal end portion of the main body, and the microelectrode comprises an array of microelectrodes disposed at a distal end portion of the main body.

In some examples, the medical implant comprises an epidermal spinal cord electrode.

In some examples, the medical implant comprises a spinal cord stimulator.

In another representative example, a method for treating a patient comprises implanting a patient-controlled analgesic device at or adjacent a surgical area of a patient. The patient-controlled analgesic device comprises a microelectrode and a control member, the microelectrode includes a conductive polymer doped with an anesthetic, and the control member is configured to allow the patient to self-administer an effective dose of the anesthetic.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A depicts a perspective view of an exemplary implantable patient-controlled analgesic device.

FIG. 1B depicts a detail view of microelectrode array of the implantable patient-controlled analgesic device of FIG. 1.

FIGS. 2A-2E depict cross-sectional views of the microelectrode array during various stages of manufacturing.

FIG. 3 schematically depicts the implantable patient-controlled analgesic device of FIG. 1 implanted at an exemplary surgical area.

FIG. 4 depicts a perspective view of another exemplary implantable patient-controlled analgesic device.

FIG. 5 depicts a perspective view of another exemplary implantable patient-controlled analgesic device.

FIG. 6 schematically depicts the implantable patient-controlled analgesic device of FIG. 5 implanted at an exemplary surgical area.

FIG. 7 depicts a table comprising several exemplary materials that can be used as a substrate for a flexible microelectrode, along with several exemplary mechanical and electrical properties of the exemplary materials.

FIG. 8 depicts an example of a resistance versus strain test apparatus for uni-axial loading.

FIGS. 9A-9E depict cross-sectional views of another exemplary microelectrode array during various stages of manufacturing.

FIG. 10 depicts an exemplary shadow mask that can be used to form a microelectrode array.

FIG. 11A depicts exemplary electrodes coated via sputtering.

FIG. 11B depicts exemplary electrodes coated via electron beam deposition.

FIG. 12 depicts an example of a flexible electrode with a layer of conductive polymer.

FIGS. 13A-13F depict cross-sectional views of another exemplary microelectrode array during various stages of manufacturing.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

Additionally, to facilitate review of the various embodiments, the following explanations of terms are provided:
Administration: To provide or give to a subject a pharmaceutical agent by any effective route.
Coated electrode: An electrode with a layer of material that partially or fully covers the conductive surface (or surfaces) of the electrode. The layer of material is itself conductive. For example, a conductive polymer can be electrodeposited on the conductive surface of an electrode to generate an electrode coated with the conductive polymer. The conductive surface of the electrode does not need to be completely coated (and in many cases is partially coated). Further, the amount of coating can vary according to the application parameters, e.g., time of use, exposure, level of signals, levels of noise, desired compound density, etc. In some instances, the drug dosage can be controlled via the surface thickness and/or through the drug solution concentration during coating. The coat or coating on a coated electrode is the layer of material that partially or fully covers the conductive surface (or surfaces) of the electrode.
Conductive polymer: An organic molecule of repeating structural units (monomers) that conducts electricity. In several embodiments, the conductive polymers provided herein can be formed by electrodeposition on an electrode. Non-limiting examples of conductive polymers for use in the embodiments disclosed herein include poly-pyrrole (PPy) and its derivatives, poly 3,4 ethylene dioxythiophene (PEDOT), polythiophene and its derivatives, and polyaniline.
Doping and Dopants: Doping is the process of oxidizing (p-doping) or reducing (n-doping) a neutral polymer and providing a counter anion or cation (the dopant). Doping can also occur during electropolymerization process, in which monomers are oxidized and polymerized into charged long chain molecules while counter ions nearby incorporated via electrostatic force. Upon doping, a conductive polymer system with a net charge of zero is produced due to the close association of the counterions with the charged conductive polymer backbone. In some examples, dopants can catalyze the polymerization of monomers during synthesis. The disclosed embodiments can, in some instances, include negatively charged nanoparticles as dopants in a conductive polymer, which alter the conductive properties of the polymer. A “doped” conductive polymer is a conductive polymer including a dopant.
Effective amount: The “effective amount” of a composition or agent is the quantity of the composition or agent sufficient to achieve a desired result.
Electrode: An electric conductor through which an electric current can pass. An electrode can also be a collector and/or emitter of an electric current. In some embodiments, an electrode is a solid and comprises a conducting metal as the conductive layer. Non-limiting examples of conducting metals include noble metals and alloys, such as stainless steel and tungsten.
Implanting: Completely or partially placing a medical device within a subject, for example, using surgical techniques. A device is partially implanted when some of the device reaches, or extends to the outside of, a subject. Implantable devices may be implanted into tissue, such as tissue adjacent a joint, more particularly the spine, for treatment of different medical conditions and for various time periods. For example, a surgical drain can be implanted for varying durations (e.g., one to four days).
Medical Implant: Any device intended to be partially or wholly introduced, inserted, or implanted within a subject's body for one or more therapeutic or prophylactic purposes, such as for restoring physiological function, alleviating symptoms associated with disease, delivering therapeutic agents, detecting changes (or levels) in the internal environment, and/or repairing or replacing or augmenting damaged or diseased organs and tissues. One non-limiting example of a medical implant is a surgical drain.

### Introduction to the Disclosed Technology

Described herein are devices and methods directed to pain management. In particular, disclosed herein are implantable patient-controlled analgesic devices (PCA devices) and related methods. The disclosed devices and methods can, for example, reduce opioid use and improve pain management following surgery.

Examples are provided throughout the present disclosure. Some of the examples describe the disclosed devices and methods in association with a particular medical procedure (e.g., spinal surgery), device (e.g., surgical drains), and/or medication (e.g., bupivacaine). It should be noted, however, that the devices and methods disclosed herein can be used in association with various other medical procedures, devices, and/or medications.

As mentioned above, opioids have been foundational to analgesia for hundreds of years. Nevertheless, opioids have widely recognized adverse side effects and addictive properties. In fact, 18.7% of neurosurgical patients are chronic opioid users.

The devices and methods herein, among other things, can reduce opioid use following surgery while optimizing pain management. The disclosed devices and methods can, for example, provide local pain relief directly to the surgical site. In some instances, the disclosed devices can be placed concurrently with the standard practice insertion of a surgical drain, thereby utilizing pre-existing protocols.

A surgical drain, which is placed in most major surgeries, remains in the surgical area for an average of 2.88 days, in this way allowing the clinician to target the most severe pain window post-surgery and optimize recovery via the disclosed devices. The disclosed devices are patient-controlled analgesia (PCA) devices. These devices can, for example, reduce overall drug dosage, as self-administration of drugs has been shown to provide maximum pain relief at a minimum dosage. Minimizing dose reduces the risk for systemic toxicity in addition to limiting side effects.

This disclosure specifically references developing flexible bioelectronics for, as one example, the application of improved post spine surgery drug delivery. It also describes the current pain relief drug delivery method and the unmet need for localized drug delivery and how it can be achieved through an implantable flexible bioelectronic (such as the PCA devices disclosed herein). A detailed outline of the flexible biocompatible substrate materials and thin film deposition techniques is discussed, identifying several relevant factors for this specific application. To ensure medical relevance and uniform drug delivery, factors such as film and substrate thickness, substrate material, and patterning technique are also disclosed.

As one example, adequate postoperative spine surgery pain management can improve functional outcomes, early post-surgical ambulation, recovery, and/or reduce the likelihood or prevent the development of chronic pain. Due to the central nervous system's complexity and fragility, oral opioids are commonly used to treat spine surgery-associated pain. Oral opioids are one of the most effective drugs used for controlling pain. However, the significant negative side effects of opioids, such as analgesic tolerance, hyperalgesia, and addiction, indicate an unmet need for improved postoperative pain management. Disclosed herein are implantable drug-loaded devices that can deliver a controlled dosage of pain medication (and/or other medication) directly to the surgical site, thereby reducing opioid use and associated side effects. As one advantage, the controlled local drug delivery of the disclosed devices, which provide a minimal systemic dose, reduce the risk of systemic toxicity by lowering the overall drug concentration while still achieving the same pain relief.

### Examples of the Disclosed Technology

As a brief overview, the disclosed implantable patient-controlled analgesic devices comprise a biocompatible drug-loaded conductive polymer that will be controlled by a microelectrode array. The array can, in some instances, be attached to a surgical drain, thereby facilitating controlled local anesthetic delivery directly to the damaged tissue through a pre-existing device. A surgical drain remains in the surgical area for a time, thereby allowing the clinician to target the most severe pain window post-surgery and optimize recovery.

Combining the disclosed implantable patient-controlled analgesic (PCA) device with a surgical drain provides one or more advantages. For example, the combination PCA-drain device is preferable to inserting an additional device or tube because it minimizes the number of foreign bodies implanted in the patient, thus simplifying the procedure and lowering infection risk. Additionally, or alternatively, by housing the drug locally on the drain, no external pumps or fluids are needed.

In some embodiments, drug delivery can be controlled through pulsatile release by applying a potential across the conductive polymer, thereby causing it to transition between oxidized/reduced states and facilitate ionic transport of the bioactive molecule. In this way, maintaining the concentration of local anesthetic within the therapeutic range can be achieved while preventing a toxic systemic concentration. As a PCA, this device can also reduce overall drug dosage, as self-administration of drugs has been shown to provide maximum pain relief at a minimum dosage.

In some embodiments, bupivacaine can be used as the anesthetic. Additionally, or alternatively, other anesthetics can be used, including nonsteroidal anti-inflammatory drugs (NSAIDs) and/or opioids.

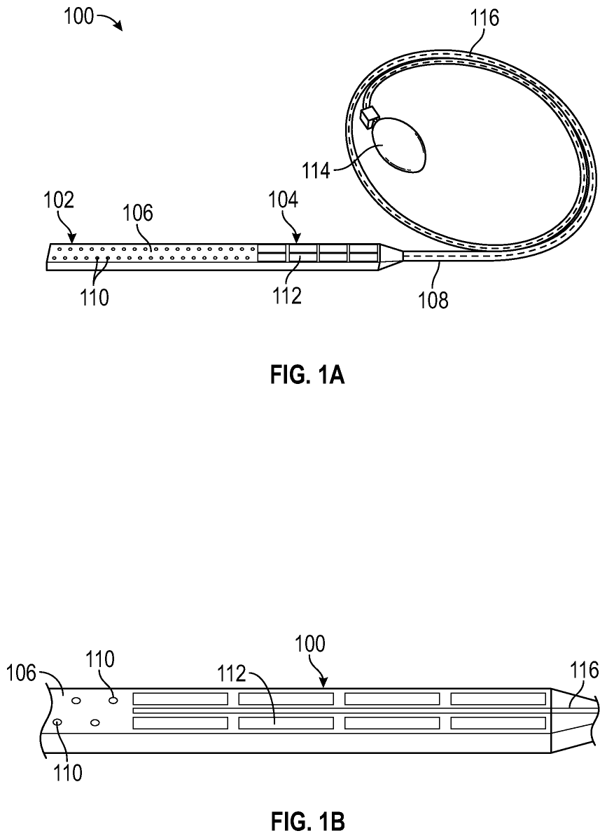

FIG. 1A depicts an implantable patient-controlled analgesic (PCA) device 100, according to one exemplary embodiment. The PCA device 100 comprises a surgical drain portion 102 and a PCA portion 104 coupled to the surgical drain portion 102. In this manner, the PCA device 100 can also be referred to as “an assembly” comprising a medical implant (e.g., a surgical drain) and the PCA device 100.

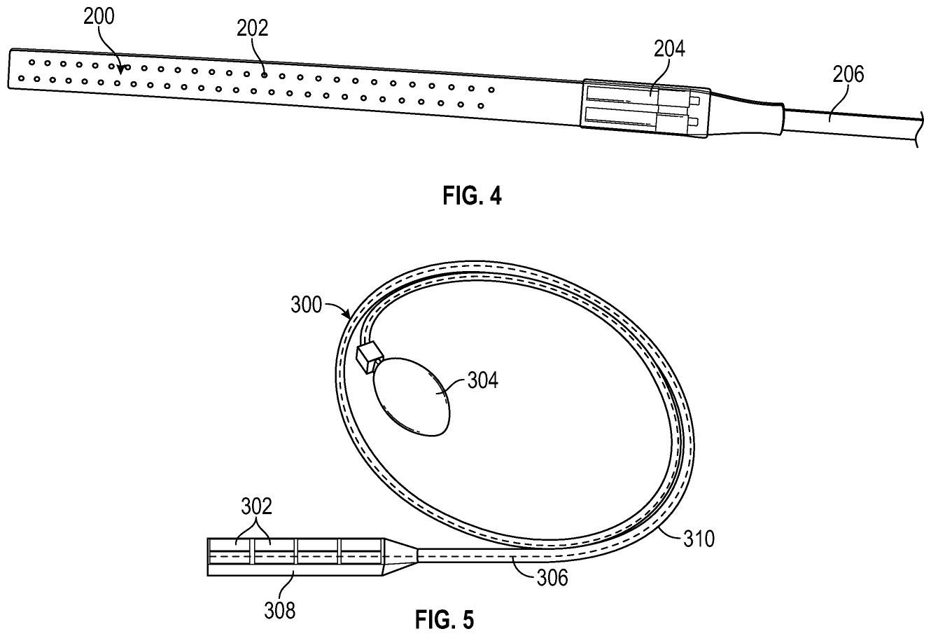

The surgical drain portion 102 of the PCA device 100 comprises a main body 106 and a conduit 108 extending from the main body 106. The main body 106 is relatively flexible and is configured to be implanted within a patient's body at a surgical area (see, e.g., FIG. 4). The main body 106 comprises one or more openings 110 (which can also be referred to as “ports”) configured to allow fluid to flow from the surgical area into the main body 106 and through the conduit 108 to a location disposed outside of the patient's body.

The PCA portion 104 of the PCA device 100 is a micro-electro-mechanical system (MEMS) comprising one or more microelectrodes, one or more control members, and one or more connection members. For example, the PCA portion 104 comprises a microelectrode array 112, a control member 114, and a connection member 116 disposed between and interconnecting the microelectrode array 112 and the control member 114. The microelectrode array 112 is coupled to the main body 106 of the surgical drain portion 102. The control member 114 is configured to be disposed outside the patient's body and to be actuated by a patient, medical professional, and/or automated system. The connection member 116 (e.g., one or more wires) is configured to extend from the control member 114 to the microelectrode array 112.

Referring still to FIG. 1A, the microelectrode array 112 of the PCA portion 104 is disposed on a proximal end portion of the main body 106 of the surgical drain portion 102, and the openings 110 are disposed on a distal end portion of the main body 106 of the surgical drain portion 102. In other embodiments, the microelectrode array 112 of the PCA portion 104 can be disposed at various other location on the main body 106 of the surgical drain portion 102 (e.g., at the distal end portion), and the openings 110 can be disposed at various other locations on the main body 106 of the surgical drain portion 102 (e.g., at the proximal end portion). In some embodiments, the microelectrode array 112 can be interspersed with the openings 110. In yet other embodiments, the microelectrode array 112 can be on a first side of the main body 106, and the opening 110 can be disposed on a second side of the main body (e.g., an opposite and/or adjacent side).

FIG. 1B depicts a detail view of the main body 106 and the microelectrode array 112.

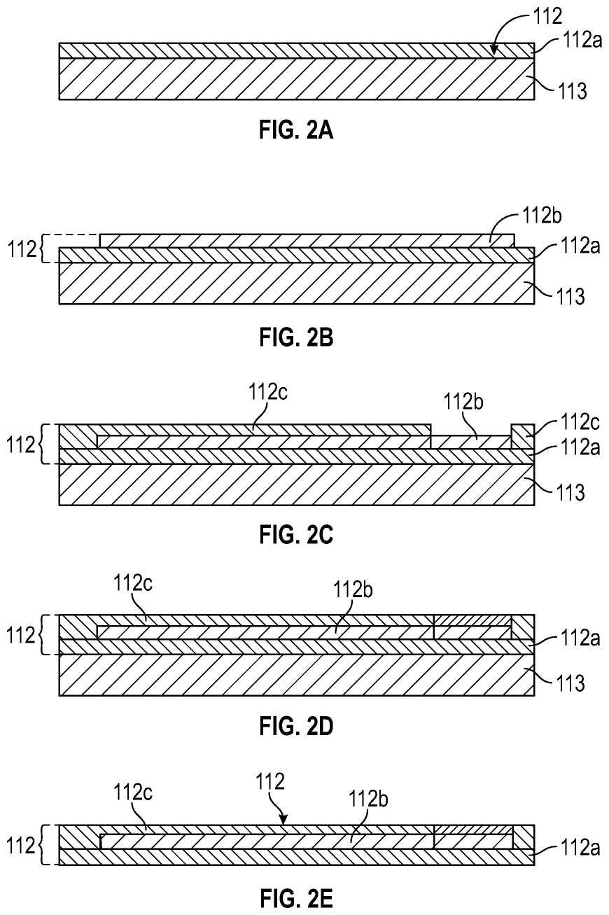

FIG. 2A-2E depict cross-sectional views of the microelectrode array 112 during an exemplary method of forming the microelectrode array 112. The microelectrode array 112 can be printed onto poly-dimethyl-siloxane (PDMS) layer 112a. This flexible substrate microelectrode can be embedded with a bupivacaine-doped conductive polymer. As depicted in FIG. 2A, PDMS can be spun onto a silicon wafer 113. As depicted in FIG. 2B, photolithography can be used to create the thin-film metal features. In particular, a thin chromium layer 112b can be placed first for better adhesion of the gold electrodes. As depicted in FIG. 2C, another PDMS layer 112c can encase the electrode features to better improve the overall electromechanical characteristics of the device. As depicted in FIG. 2D, a doped conductive polymer 112d can be deposited after the electrodes have been fully fabricated. As depicted in FIG. 2E, the microelectrode array 112 can be removed from the silicon wafer 113.

Once fabricated, the microelectrode array 112 can be coupled to a medical implant (e.g., a surgical drain) in various ways. For example, the microelectrode array 112 can be coupled to a medical implant using biocompatible adhesive. Some exemplary biocompatible adhesives include: silicone sealant, poly-methyl-methacrylate (PMMA), cyanoacrylate, enzymatic glues (CelTak, matrigel collagen), and UV-curable adhesives.

The drug-loaded conductive polymer can be created through electrosynthesis. Poly-pyrrole (PPy) is one exemplary material that can be used as the conductive polymer due to its biocompatibility, stability, and easy electrosynthesis. The PPy film can be composed of pyrrole, deionized water, and sodium dodecylbenzenesulfonate (NaDBS). In other instances, one or more other surfactants can be used. The film deposition can, in some instances, be a two-layer process; the first being a drug-free adhesion layer and the second being a bulk drug-containing layer.

The conductive polymer can be doped with one or more dopants. Exemplary dopants include: bupivacaine, NSAIDs, opioids, and/or other anesthetics or analgesics. In other embodiments, other types of dopants (i.e., besides anesthetics) can be used.

In some embodiments, one or more electrodes can be doped separately from one or more other electrodes. For example, one or more electrodes can be doped in a first process and comprise a first drug (e.g., bupivacaine), and one or more other electrodes can be doped in a second process and comprise a second drug (e.g., an opioid). As another example, one or more electrodes can be doped in a first process and comprise a first drug concentration (e.g., bupivacaine concentration 1), and one or more other electrodes can be doped in a second process and comprise a second drug concentration (e.g., bupivacaine concentration 2).

As mentioned above, the control member 114 is configured to be disposed outside the patient's body and to be actuated by a patient, medical professional, and/or automated system. The control member can, for example, allow the patient to administer an effective amount of the medication directly to the source of the pain. The control member 114 can include one or more buttons, dials, actuators, etc. configured to send an electrical current to the microelectrode, which results in a dosage of medication being released from the microelectrode array into the patient's body.

The connection member 116 (e.g., one or more wires) is configured to extend from the control member 114 to the microelectrode array 112. In some embodiments, the connection member 116 can extend through the same tube or shaft as the drainage tube of the surgical drain portion. For example, a tube can comprise a drainage lumen and a connection member lumen. In other embodiments, the connection member 116 can be housed in a separate shaft that is adjacent and/or coupled to the drainage tube of the surgical drain.

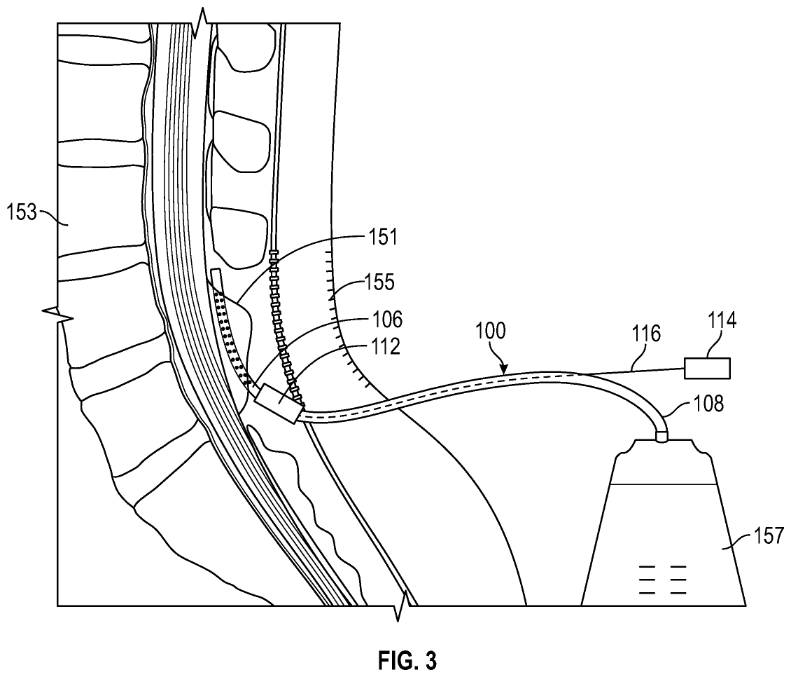

FIG. 3 schematically depicts the PCA device 100 implanted with a patient's body at an exemplary surgical area 151 (e.g., adjacent a patient's spine 153). The surgical area 151 can be closed around the PCA device 100 via sutures 155 and/or other skin closure means. The connection member 116 of the PCA device 100 can exit the conduit 108 (e.g., at a location outside of the patient's body). The patient (or other person or an automated system) can access the control member 114 to administer the medication at the surgical location when needed and/or as prescribed. In the exampled depicted in FIG. 3, the PCA device 100 is integrated into a surgical drain device. Drainage can, for example, flow from the surgical area into the openings 110 (see FIG. 1B), through the main body 106, through the conduit 108, and to a drainage bag 157 disposed outside of the patient's body.

FIG. 4 depicts a portion of an implantable patient-controlled analgesic device 200, according to another exemplary embodiment. The PCA device 200 comprises a surgical drain portion 202 and a microelectrode portion 204. The PCA device 200 further comprises a conduit 206. The PCA device 200 can be configured in a manner similar to the PCA device 100, as described above. One difference between the PCA device 200 and the PCA device 100 is that the microelectrode portion 204 of the PCA device 200 is spaced farther from the openings of the surgical drain portion 202 compared to the PCA device 100.

FIG. 5 depicts an implantable patient-controlled analgesic device 300. The PCA device 300 comprises a microelectrode array 302, a control member 304, and a connection member 306 disposed between and interconnecting the microelectrode array 302 and the control member 304. The microelectrode array 302 is coupled to a main body portion 308. The control member 304 is configured to be disposed outside the patient's body and to be actuated by a patient, medical professional, and/or automated system. The connection member 306 (e.g., one or more wires) is configured to extend from the control member 304 to the microelectrode array 302. In some examples, the connection member 306 can be housed within the main body portion and/or a cover 310 (e.g., a sleeve or tube), which can, among other things, protect and/or insulate the connection member 306.

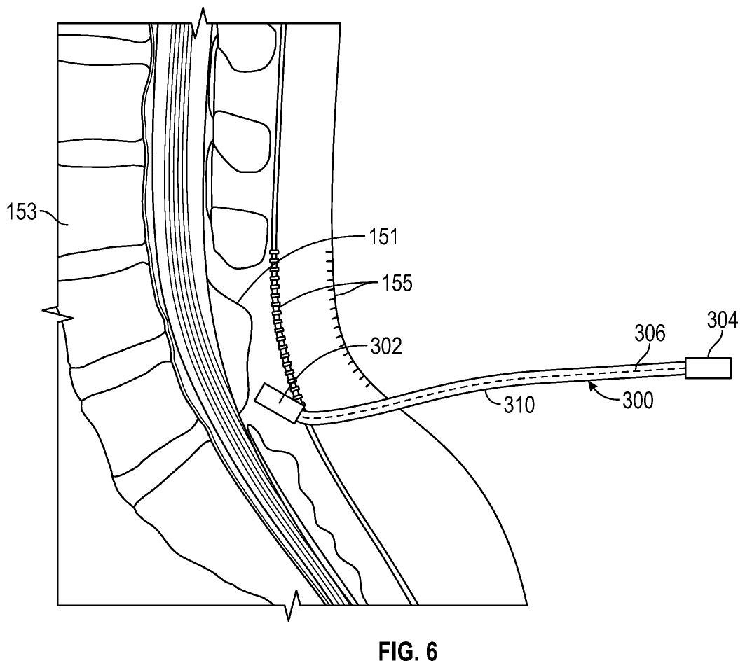

FIG. 6 depicts the PCA device 300 implanted with a patient's body at the exemplary surgical area 151 (e.g., adjacent the patient's spine 153). The surgical area 151 can be closed around the PCA device 300 via sutures 155 and/or other skin closure means. The connection member 306 extend from the main body portion 308 disposed within the patient's body to the control member 304 disposed outside of the patient's body. The patient (or other person or an automated system) can access the control member 304 to administer the medication at the surgical location when needed and/or as prescribed.

Additional information and examples regarding the disclosed devices and methods are provided below. The embodiments described and their respective figures are examples are not intended to limit the scope of the disclosure.

Conductive polymers can load and release a bioactive molecule by applying a potential causing it to transition between oxidized/reduced state facilitating drug release. It is possible to characterize the amount of drug released per the electric potential.

Flexible electrodes can incorporate the doped conductive polymer and be implemented to interface with neural tissue. Flexible electronics can conform to unique topographies, are lightweight, and can be stretched repeatedly without fatigue. In some instances, during mechanical deformation, the electrical properties can become unstable and fatigue before mechanical failure is reached. Determining the effect of mechanical deformation on the electrical characteristic can help ensure uniform drug release from the conductive polymer. The degradation of electrical properties during manufacturing and induced strain is discussed herein.

The residual and induced stresses can, for example, help to understand the electrical characteristics. The residual stress can be understood through investigating film and substrate material interaction and deposition techniques. They can be caused by the film to substrate interface delamination and fracture. Delamination can be caused by thermal mismatch or surface flaws, which can cause compressive and tensile stress, creating buckling, wrinkling, or channel cracking. Some of these fractures can be controlled through deposition parameters. The induced stresses can be determined by understanding the dynamic implantable surgical area environment that could generate cyclic strains.

The substrate and film material and thickness with the corresponding deposition techniques can determine the structure that will occur and the introduced residual stresses. The material's behavior can be dictated by thermal and elastic mismatch and a larger impurity density. These stresses can affect thin film adhesion, crystalline defects, and film topography. Each factor can contribute to the electrical characteristics of the system. Thin films can have a smaller grain size than bulk materials and have a high degree of texturing from processing parameters. One identified fatigue that leads to electrical instability is the initiation of microcracks at voids or impurity particles during cyclic repeated deformation. Due to the dynamic environment in which the device can be implanted, it can be vital to understand the stresses within the film before and after deformation.

Some exemplary biocompatible flexible substrates materials for implantable devices include polyimide, Parylene-C, and Polydimethylsiloxane (PDMS). Each material comprises unique mechanical, electrical, and/or handling/processability characteristics. The material can be selected for dynamic tissue interfacing.

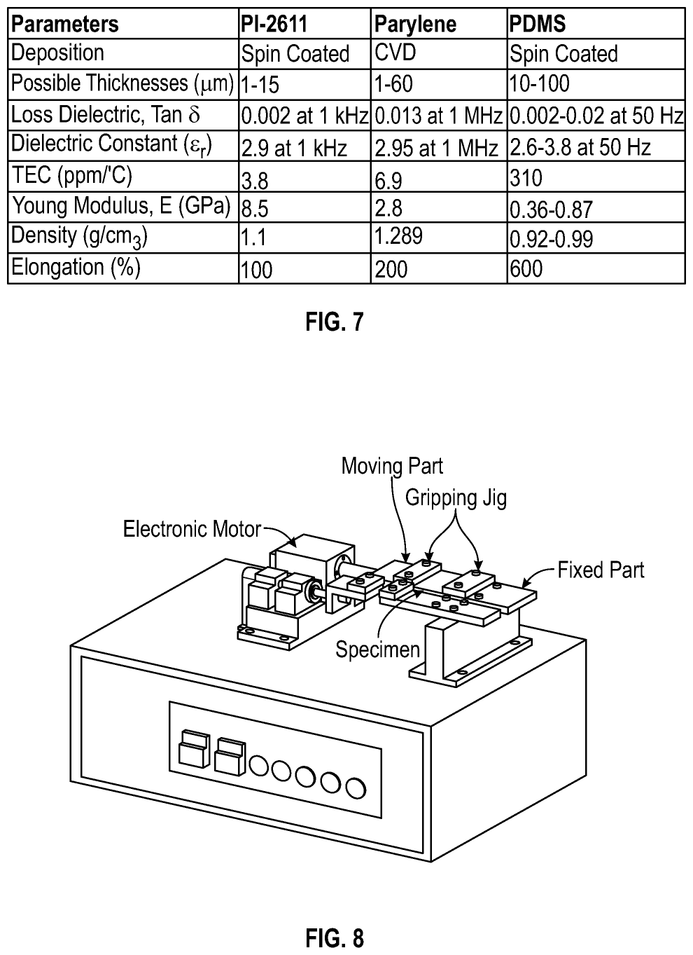

As one example, polyimide is a well-suited material for integrating electrodes due to its excellent metal adhesion. As another example, Parylene-C also has an excellent metal adhesion and provides good processability. PDMS has an excellent elasticity property. The table depicted in FIG. 7 provides several mechanical and electrical properties of exemplary materials.

A PCA device can be flexible, conformable and accommodating to the dynamic environment. PDMS is an attractive polymeric matrix because of its ability to conform and interface with neural tissue. Polyimide is also a suitable substrate for this application. The deposition techniques and potential solutions for reduced stress on PDMS are further discussed below.

During deformation, electrical instability for Au films on PDMS relies, at least in part, on the initial morphology of the metal on PDMS. Deposition techniques approved for various medical implants include sputtering, electron beam, and thermal evaporation techniques. Due to PDMS's relatively large thermal expansion coefficient, sputtering and electron beam deposition can help reduce thermal mismatch and its corresponding stress. In some instances, one issue with depositing thin films on polymers is that metals' adhesion can be insufficient. Adhesion can be enhanced through surface treatment and a thin layer of Cr/Ti. Additional metals can, however, lead to increased stiffness of the multilayered thin film.

Gold film thickness is another parameter that can be controlled to help maintain electrical stability. The deposited thickness can determine the electrical characteristics and mechanical robustness that is desired. For example, evaporation of 5000 Å thick gold film onto the PDMS layer is the max gold deposition thickness for a single evaporation step reducing residual stress during deposition. When exceeding this thickness, there can be an increase of microcracks, reducing the electrodes' conductivity. Each of these parameters can be taken into consideration to reduce the amount of residual stress.

Patterning techniques for PDMS include shadow masking to transfer patterns, planarization, and photolithography. Each of these techniques can bypass solvents' use to avoid PDMS swelling, which can initiate extrinsic stress. Patterning through shadow masking and planarization can provide good results but may be limited to a minimum feature size of 100 μm in some instances. Photolithography can create a minimum feature size of 10 μm but can, in some implementations, be an intricate process regarding photoresist choice, thickness, baking procedure, and removal.

Electrical stability can depend on the microstructure of the gold film during stretching. The cracks that are formed during stretching can change the electrical resistance of the thin film. The gold film on PDMS can rely on the electrical current's percolation through a built-in network of gold ligaments and microcrack distribution. When the system is stretched or compressed, the polymer substrate carries most of the deformation while the gold creates microcracks between the gold ligaments. Lateral and longitudinal cracks appear during compression and tension.

In some examples, lateral cracks can significantly increase the electrode's resistance, while the longitudinal cracks have little influence. A resistance versus strain test apparatus for uni-axial loading is depicted in FIG. 8. The apparatus can have two clamps to secure the PDMS electrodes and stretch at a known rate while using a two-probe method to measure the electrodes' electrical resistance. The desired strain can range from 0.5%-30% to identify the point of electrical failure.

The evolution of surface microcrack density can be quantitatively established for both the tension and compression zones. It has been determined that there is a slight increase in electrical resistance during initial strain due to microcracks. However, gradually, electrical resistance increased then abruptly decreased due to the continuous growth of microcracks during deformation. The microcracks and their interaction with electrical resistances can, in some instances, result in non-uniform drug delivery depending on the environment.

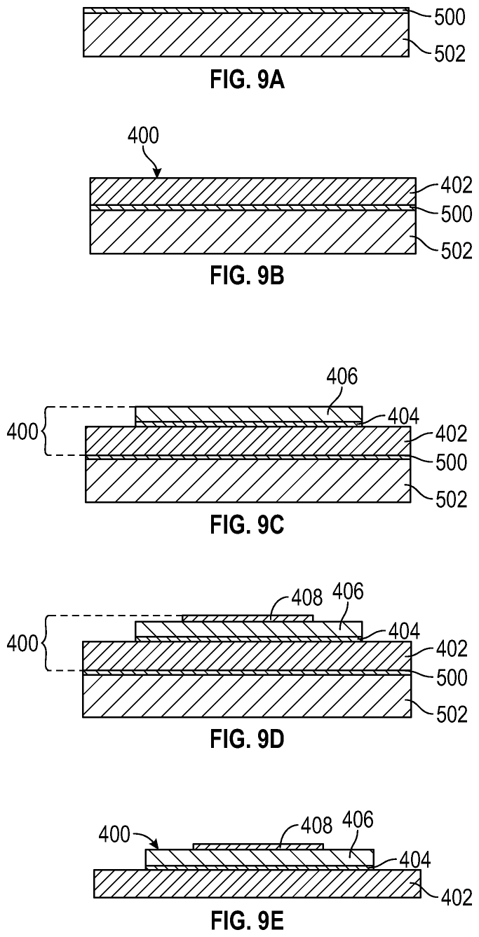

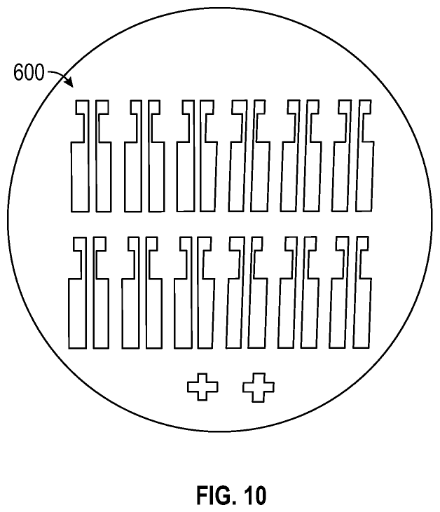

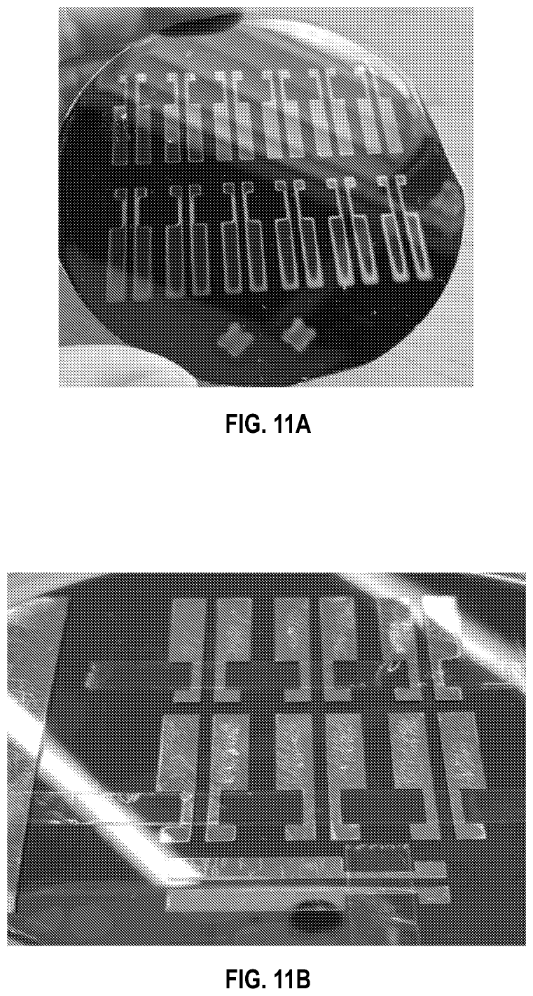

Another exemplary method of forming a flexible microelectrode 400 is depicted in FIGS. 9A-9E. Referring to FIG. 9A, an antiadhesion layer 500 (e.g., 5 nm of Parylene-C) is coated onto a silicon wafer 502. The antiadhesion layer 500 can help with removal of the flexible microelectrode 400 after the fabrication process is complete. An elastomer layer 402 (e.g., PDMS) can be applied to the antiadhesion layer 500, as depicted in FIG. 9B. As one example, PDMS can be mixed (e.g., Sylgard 184©, Dow Corning) at 10:1 ratio of PDMS prepolymer to curing agent ratio and defoamed. The mixture can be spin-coated onto the silicon wafer. Various speeds (e.g., 1000-500 rpm) can be used to create various thicknesses (e.g., from 70 μm-200 μm). The PDMS can be cured in a vacuum (e.g., at 60 C for 24 hrs.). Referring now to FIG. 9C, the elastomer layer 402 can be coated with a Chromium (Cr) layer 404 (e.g., 5 nm) and a Gold (Au) layer 406 (e.g., 49 nm). In one or more embodiments, a shadow mask 600 (FIG. 10) can be attached to the elastomer layer 402 prior to applying one or more layers thereon (e.g., the Cr layer 404 and/or the Au layer 406). The electrodes can be coated in various ways (e.g., using sputtering and/or electron beam deposition). FIG. 11A depicts exemplary electrodes coated via sputtering, and FIG. 11B depicts exemplary electrodes coated via electron beam deposition. Referring now to FIG. 9D, a polyimide layer 408 (e.g., polyimide tape) is attached to insulates the leads. In some examples, the insulation process can create an electrode embedded in elastomer layer 402 in lieu of or in addition to using polyimide tape. Referring to FIG. 9E, the flexible microelectrode 400 can be removed from the wafer.

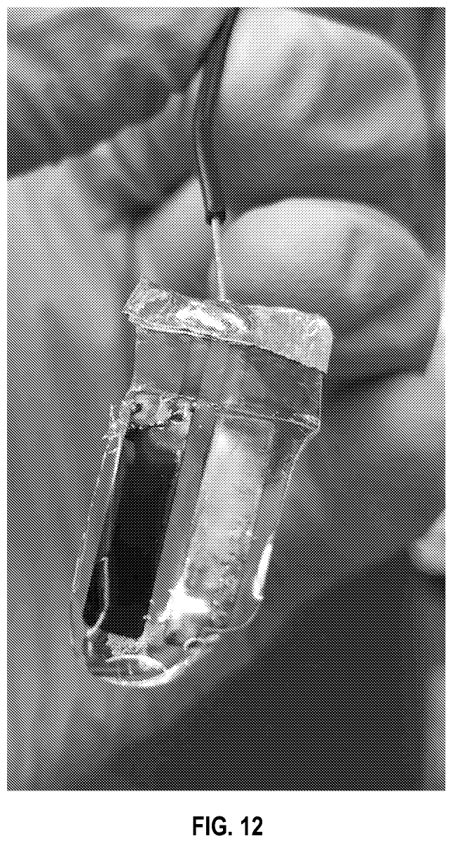

FIG. 12 depicts an example of a flexible electrode with a layer of conductive polymer. The flexible electrode can, for example, be used with any of the PCA devices disclosed herein. For example, the flexible electrode can be used with the PCA device 200 in lieu of the microelectrode portion 204.

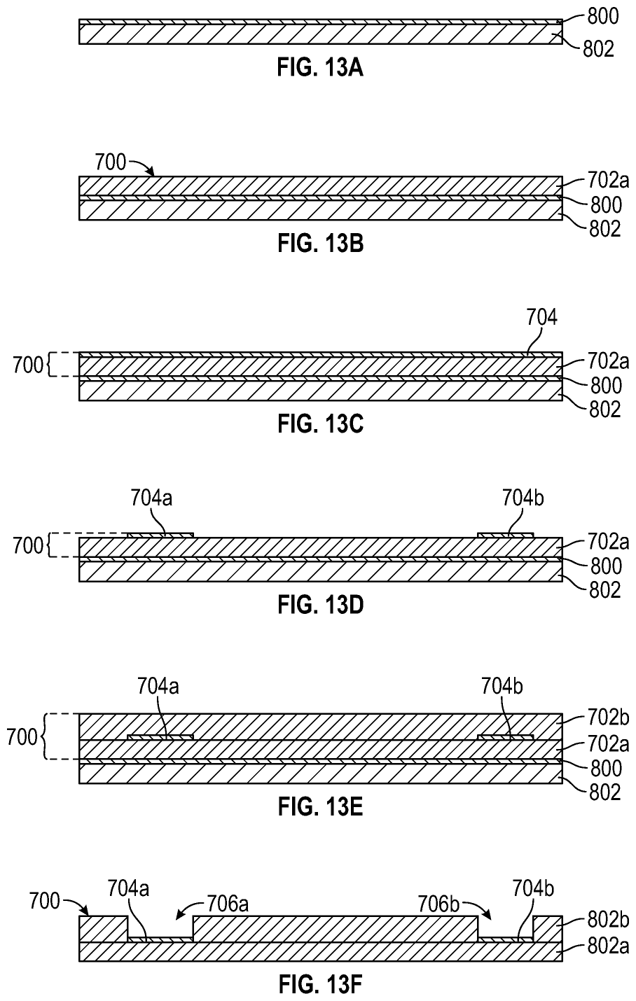

FIGS. 13A-13F depict another exemplary method of forming a flexible microelectrode 700. Referring to FIG. 13A, a release layer 800 (e.g., Cr) is coated onto a silicon wafer 802. The release layer 800 can help with removal of the flexible microelectrode 700 after the fabrication process is complete. A first polymeric layer 702a (e.g., polyimide) can be applied to the release layer 800, as depicted in FIG. 13B. The electrode layer 704 can be deposited onto the first polymeric layer 702a, as depicted in FIG. 13C. The electrode layer 704 can be formed of various materials. For example, in some implementations, the electrode can comprise titanium, platinum, and/or gold. Referring to FIG. 13D, photolithography can be used to form one or more electrodes (e.g., the electrodes 704a and 704b) from the electrode layer 704 (FIG. 13C). A second polymeric layer 702b (e.g., polyimide) can be applied onto the electrodes 704a and 704b and the first polymeric layer 702a, as depicted in FIG. 13E. Referring now to FIG. 13F, electrode vias 706a and 706b can be formed in the second polymeric layer 702b, and the flexible microelectrode 700 (which can be part of a microelectrode array) can be removed from the release layer 800 and the silicon wafer 802.

The disclosed technology can, for example, improve post-surgical care through controlled local drug delivery. While current pain management methods are inadequate for optimal patient care, the technology disclosed herein can, among other things, allow for individual-dependent drug dosing, thereby providing pain relief and personalized medicine while reducing the risk for future opioid dependency.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. An implantable patient-controlled analgesic device comprising a microelectrode, a control member, and a connections member. The microelectrode comprises a conductive polymer doped with a dopant, wherein the microelectrode is configured to be implanted within a patient's body. The control member is configured to control the microelectrode via an electrical current. The connection member is coupled to the microelectrode and the control member such that the electrical current can flow from the control member to the microelectrode.
Example 2. The implantable patient-controlled analgesic device of any example herein, and particularly example 1, wherein the dopant comprises one or more of bupivacaine, a non-steroidal anti-inflammatory drug, an opioid, and/or or other anesthetic or analgesic.
Example 3. The implantable patient-controlled analgesic device of any example herein, and particularly either example 1 or example 2, further comprising a main body on which the microelectrode is disposed.
Example 4. The implantable patient-controlled analgesic device of any example herein, and particularly any one of examples 1-3, wherein the microelectrode is one microelectrode of a plurality of microelectrodes arranged in an array.
Example 5. The implantable patient-controlled analgesic device of any example herein, and particularly any one of examples 1-4, further comprising a surgical drain portion, wherein the surgical drain portion comprises a plurality of openings and a conduit, wherein the conduit is in fluidic communication with the plurality of openings such that fluid can flow into the openings and through the conduit.
Example 6. The implantable patient-controlled analgesic device of any example herein, and particularly example 5, wherein the surgical drain portion is disposed distal relative to the microelectrode.
Example 7. The implantable patient-controlled analgesic device of any example herein, and particularly example 5, wherein the surgical drain portion is disposed proximal relative to the microelectrode.
Example 8. The implantable patient-controlled analgesic device of any example herein, and particularly any one of examples 1-7, wherein the microelectrode comprises polyimide.
Example 9. The implantable patient-controlled analgesic device of any example herein, and particularly any one of examples 1-7, wherein the microelectrode comprises poly-dimethyl-siloxane.
Example 10. The implantable patient-controlled analgesic device of any example herein, and particularly any one of examples 1-9, wherein the conductive polymer comprises poly-pyrrole.
Example 11. The implantable patient-controlled analgesic device of any example herein, and particularly any one of examples 1-10, wherein the microelectrode comprises titanium.
Example 12. The implantable patient-controlled analgesic device of any example herein, and particularly any one of examples 1-11, wherein the microelectrode comprises platinum.
Example 13. The implantable patient-controlled analgesic device of any example herein, and particularly any one of examples 1-12, wherein the microelectrode comprises gold.
Example 14. The implantable patient-controlled analgesic device of any example herein, and particularly any one of examples 1-13, wherein the microelectrode comprises chromium.
Example 15. An assembly comprising a medical implant and an implantable patient-controlled analgesic device coupled to the medical implant. The implantable patient-controlled analgesic device coupled to the medical implant includes a microelectrode comprising a conductive polymer doped with a dopant, wherein the microelectrode is configured to be implanted within a patient's body. The control member is configured to control the microelectrode via an electrical current. The connection member is coupled to the microelectrode and the control member such that the electrical current can flow from the control member to the microelectrode.
Example 16. The assembly of any example herein, and particularly example 15, wherein the medical implant comprises a surgical drain having a main body with a plurality of openings disposed at a distal end portion of the main body, and wherein the microelectrode comprises an array of microelectrodes disposed at a proximal end portion of the main body.
Example 17. The assembly of any example herein, and particularly example 15, wherein the medical implant comprises a surgical drain having a main body with a plurality of openings disposed at a proximal end portion of the main body, and wherein the microelectrode comprises an array of microelectrodes disposed at a distal end portion of the main body.
Example 18. The assembly of any example herein, and particularly example 15, wherein the medical implant comprises an epidermal spinal cord electrode.
Example 19. The assembly of any example herein, and particularly example 15, wherein the medical implant comprises a spinal cord stimulator.
Example 20. A method for treating a patient, comprising implanting a patient-controlled analgesic device at or adjacent a surgical area of a patient. The patient-controlled analgesic device comprises a microelectrode and a control member, the microelectrode includes a conductive polymer doped with an anesthetic, and the control member is configured to allow the patient to self-administer an effective dose of the anesthetic.

In view of the many possible embodiments to which the principles of the disclosure may be applied, it should be recognized that the illustrated embodiments are only examples and should not be taken as limiting the scope of the claims. Rather, the scope of the claimed subject matter is defined by the following claims and their equivalents.

1. An implantable patient-controlled analgesic device comprising:
a microelectrode comprising a conductive polymer doped with a dopant, wherein the microelectrode is configured to be implanted within a patient's body;
a control member configured to control the microelectrode via an electrical current; and
a connection member coupled to the microelectrode and the control member such that the electrical current can flow from the control member to the microelectrode.

2. The implantable patient-controlled analgesic device of claim 1, wherein the dopant comprises one or more of bupivacaine, a non-steroidal anti-inflammatory drug, an opioid, and/or other anesthetic or analgesic.

3. The implantable patient-controlled analgesic device of claim 1, further comprising a main body on which the microelectrode is disposed.

4. The implantable patient-controlled analgesic device of claim 1, wherein the microelectrode is one microelectrode of a plurality of microelectrodes arranged in an array.

5. The implantable patient-controlled analgesic device of claim 1, further comprising a surgical drain portion, wherein the surgical drain portion comprises a plurality of openings and a conduit, wherein the conduit is in fluidic communication with the plurality of openings such that fluid can flow into the openings and through the conduit.

6. The implantable patient-controlled analgesic device of claim 5, wherein the surgical drain portion is disposed distal relative to the microelectrode.

7. The implantable patient-controlled analgesic device of claim 5, wherein the surgical drain portion is disposed proximal relative to the microelectrode.

8. The implantable patient-controlled analgesic device of claim 1, wherein the microelectrode comprises polyimide.

9. The implantable patient-controlled analgesic device of claim 1, wherein the microelectrode comprises poly-dimethyl-siloxane.

10. The implantable patient-controlled analgesic device of claim 1, wherein the conductive polymer comprises poly-pyrrole.

11. The implantable patient-controlled analgesic device of claim 1, wherein the microelectrode comprises titanium.

12. The implantable patient-controlled analgesic device of claim 1, wherein the microelectrode comprises platinum.

13. The implantable patient-controlled analgesic device of claim 1, wherein the microelectrode comprises gold.

14. The implantable patient-controlled analgesic device of claim 1, wherein the microelectrode comprises chromium.

15. An assembly comprising:
a medical implant; and
an implantable patient-controlled analgesic device coupled to the medical implant and comprising:
a microelectrode comprising a conductive polymer doped with a dopant, wherein the microelectrode is configured to be implanted within a patient's body;
a control member configured to control the microelectrode via an electrical current; and
a connection member coupled to the microelectrode and the control member such that the electrical current can flow from the control member to the microelectrode.

16. The assembly of claim 15, wherein the medical implant comprises a surgical drain having a main body with a plurality of openings disposed at a distal end portion of the main body, and wherein the microelectrode comprises an array of microelectrodes disposed at a proximal end portion of the main body.

17. The assembly of claim 15, wherein the medical implant comprises a surgical drain having a main body with a plurality of openings disposed at a proximal end portion of the main body, and wherein the microelectrode comprises an array of microelectrodes disposed at a distal end portion of the main body.

18. The assembly of claim 15, wherein the medical implant comprises an epidermal spinal cord electrode.

19. The assembly of claim 15, wherein the medical implant comprises a spinal cord stimulator.

20. A method for treating a patient, comprising:
implanting a patient-controlled analgesic device at or adjacent a surgical area of a patient, wherein the patient-controlled analgesic device comprises a microelectrode and a control member, wherein the microelectrode includes a conductive polymer doped with an anesthetic, and wherein the control member is configured to allow the patient to self-administer an effective dose of the anesthetic.
